(19) United States
(12) Patent Application Publication
(10) Pub. No.: US 2025/0194950 A1
(43) Pub. Date: Jun. 19, 2025

(54) SYSTEM FOR THE BIOMECHANICAL STUDY OF THE MOVEMENT OF A PERSON

(71) Applicant: Eduardo Sanchez-Osorio Nicolás, Barcelona (ES)

(72) Inventor: Eduardo Sanchez-Osorio Nicolás, Barcelona (ES)

(21) Appl. No.: 18/832,569

(22) PCT Filed: Sep. 8, 2020
(86) PCT No.: PCT/ES2020/000041

§ 371 Date: Jul. 24, 2024

(30) Foreign Application Priority Data
Aug. 22, 2020 (ES) U202000416

(51) Int. Cl. A61B 5/103; A61B 5/00; A61B 5/107; A61B 5/11; G06T 7/00

(52) U.S. Cl. CPC A61B 5/1038; A61B 5/1071; A61B 5/1128; A61B 5/6895; A61B 5/742; G06T 7/0012; G06T 2207/10016; G06T 2207/30196

(57) ABSTRACT

A system for the biomechanical study of the movement of a person having: a treadmill with a pressure platform that obtains real-time baropodometric images of the person's tread; a video capturing device for the real-time video capture of the position of the person's body while exercising on the treadmill; a computer to which are connected: the video capture device, the pressure platform and a user interface for a specialist in charge of controlling the mode of execution of the software and the information on the person displayed on a screen: The computer incorporates a smart software with a number of algorithms that process the information provided by the video capture device, process the baropodometric images, and provide the analytical data for the personalized biomechanical study of the person.

### CROSS REFERENCE TO RELATED APPLICATION

This application is a national stage entry of PCT/Es2020/000041 filed Sep. 8, 2020, under the International Convention and claiming priority over Spain Patent Application No. U202000416 filed Aug. 22, 2020.

### FIELD OF THE INVENTION

The present invention relates to a system for evaluating the biomechanics of the gait of an individual while walking, running or performing specific sports gestures; and which has a computer system with smart software that measures and parameterizes in real time the dynamic behavior of the individual's body and the pressure exerted by the feet of said individual on a support surface. during practice of an exercise, automatically makes a diagnosis and treatment based on personalized templates extracted from the data obtained from the evaluation performed. This system is applicable in the fields of biomechanics, podiatry, sports medicine, physiotherapy, retrofit and physical preparation.

### PRIOR ART

Currently, biomechanical studies consisting of the analysis of the way of stepping are widely known. These studies are carried out by observing the different support and pressure zones of the feet on a surface.

When some type of anomaly is detected in the tread, the adopted solution is usually the use of corrective insoles, However, the current technique does not comprehensively analyze the movements of the body assembly during the practice of the exercise, since in the best case an observation is made to the individual view of the individual while performing an exercise, which prevents determining, analytically, which are the movements of the body causing the defective tread and therefore. correcting the cause causing said defective tread.

Other techniques for observing the movements of the individual during the practice of an exercise require the use of specific markers and the use of a calibrated space to determine the position of the individual. These techniques do not make it possible to automatically make a biomechanical diagnosis and relate it to the lesions that the individual is suffering or can suffer, in order to prevent them.

The applicant of the invention does not know the existence of the current podiatry of any dynamic assessment system, capable of evaluating and measuring the kinetics and kinematics of the human body in real time. Up until now, it is based on the experience and observation power of the eye of the visitor specialist, but no numerical results that valuable the observation are obtained.

### EXPLANATION OF THE INVENTION

The system for the biomechanical study of the movement of an individual, object of this invention, allows performing the three-dimensional study of the whole body of an individual in real time, and analyze the kinetics and kinematics of the individual both to maintain the balance in the standing and the ambulation and the stroke. This system also allows analyzing the biomechanics of the specific sports gestures of different sports, for example: soccer, basketball, tennis, padel, cycling, etc.

This system is based on the union of the three-dimensional vision concept and the continuous monitoring of plantar pressures in real time in order to be able to monitor and parameterize the movement of the body. All this is integrated in a system capable of working autonomously, following working protocols validated by ergo dynamics.

This system is provided with intelligence to be able to automatically diagnose different pathologies in the process of walking of the human body, studying the kinematic and dynamic movement involved in the gait process and/or stroke, comparing the data obtained with a large database of fruit of a long experience; while other technologies focus only on the study of the lower limbs (feet) of the human body.

According to the invention, this system comprises:
a treadmill incorporating a pressure platform provided with a high-resolution pressure sensor, which obtains real-time high-resolution baropodometric images of the individual's tread while walking or running on said treadmill belt;
means for the real-time video capture of the posture of the body of the individual during the practice of the exercise on the treadmill;
a computer equipment to which they are connected: the video capture means and the pressure platform, and a user interface for a specialist responsible for controlling the mode of execution of the software and the information of the software. individual displayed on a screen; said computer equipment has smart software that includes algorithms that:
process the information provided by the video capture means, obtains data from positions, angles, speeds and accelerations of the movements of the individual in the three planes of the space and compares them to ranges of values predetermined, considered normal, deficient, optimal and pathological, determining those movements of the individual that come out of normal.
process the baropodometric images provided by the pressure platform, dynamically analyzes the pressure distribution over time and with the entire extracted tread sequence, discard the rams that do not follow the normal pattern of the sequence, and obtains a medium pressure distribution model.
providing the analytical data for the personalized biomechanical study of the individual, identifying biomechanical alterations that are the cause of a lesion, or that can pose a risk of injury to the individual, and optionally designed a totally personalized treatment by means of templates suitable for correcting the detected alterations.

According to the invention, the real-time video capture cameras may comprise one or more RGB-D vision sensors capable of measuring the depth to which the image objects are, or multiple fixed and calibrated sensors.

The video capture chambers employed are suitable and capable of delivering from 60 to 90 fps, i.e. two to three times what is capable of viewing the human eye. This makes it possible to see movements focused on detecting anomalies of the individual's gait.

The processing of the images makes it possible to process and analyze in real time the initial (static) position of the individual and their kinematic and dynamic evolution during exercise practice and to arrange in real time the evolution of the angles articular, angular velocity and position in the 3 planes of the space of the most relevant anatomical references such as knees, hips, spinal column.

The software responsible for processing the images provided by the vision sensors comprises an algorithm that records the clouds of 3D points in a single point cloud and an optimization algorithm that extracts for each image one representation of the skeleton of the individual.

The processing of said images eventually comprises the application of a smoothing or smoothing technique, the post-processing of the sequence of skeletons in order to eliminate estimation noise of the independent frameworks and the processing for obtaining the data relating to the movements: positions, angles, linear or angular velocities and linear or angular accelerations in the 3 planes of the space.

The system allows each metric to be configured independently from the user interface for the specialist, establishing the ranges of normal/deficient/optimal/pathological values from the point of view of interest or physician. These ranges are used to detect points of interest in the sequence of images in which the individual performs movements that come out of normal.

The system is configured to allow two modes of operation:
a streaming/recording mode, in which the data is sent to the user interface, while being recorded on a hard disk of the system, and
a reproduction mode, in which the data stored in a user interface is loaded for scanning.

The system also comprises a user interface for the individual showing the video and the posture, the pressure image of the feet, and a series of indicators with the angles that the visitor specialist decides.

The processing of the baropodometric images comprises the application of a local optimization algorithm at sub-pixel level to temporally align the sequence of said baropodometric images, determining the displacement between consecutive frames; this information being used to extract the historical image of the steps, displaying in the space the entire temporal sequence.

The capture of the baropodometric images allows obtaining data that would be impossible to collect on a static platform of reduced size, and to correlate the mechanism to any level, with the behavior of the foot and the balance of the individual at each moment during the practice of the exercise.

In addition to providing all analytical data for the custom biomechanical study and adjusted to the age and activity level of the individual, the system is able to identify biomechanical alterations that are the cause of a lesion, or which may pose a risk of injury to the individual.

The system itself is designed so that through the data obtained it can reach a biomechanical diagnosis and design the best fully customized treatment (templates) for the alterations.

One of the characteristics of the system is that it combines all three dimensions: length, width and depth of the 3D technology, with the dimension of the time, so that data is obtained in space and real time, this makes it possible to have all the information at the instant and determine the plan of action to follow immediately.

### BRIEF DESCRIPTION OF THE CONTENTS OF THE DRAWINGS

To complement the description being made and in order to facilitate the understanding of the features of the invention, a set of drawings is attached to the present specification in which, for illustrative and non-limiting purposes, the following has been represented:

FIG. 1 shows a schematic of the system for the biomechanical study of the movement of an individual, according to the invention.

### DETAILED EXPOSURE OF EMBODIMENTS OF THE INVENTION

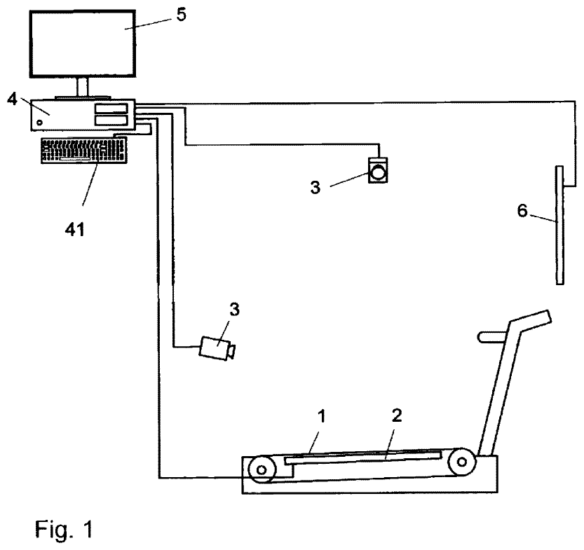

In the example of embodiment shown in FIG. 1, the components of the system of the invention have been schematically represented, comprising: —a treadmill (1) incorporating a pressure platform (2) provided with a high-resolution pressure sensor, which obtains real-time baropodometric images of the tread of the individual object of the study while walking or running on said treadmill; —means (3) for capturing video (3) in real time of the posture of the body of the individual during the practice of the exercise on the treadmill and, —a computer device (4) provided with intelligent software and to which said video capture means (2) and said pressure platform (3) are connected.

The video capture means (3) are constituted in this case by cameras with fixed and calibrated sensors, and suitable for delivering between 60 and 90 fps (frames per second).

The system comprises a user interface (41) for a visitor specialist responsible for controlling the mode of execution of the software of the computer equipment and the information of the individual displayed on a screen (5) and an interface user (6) for the individual, represented by a screen showing the video and the posture of the individual during the practice of the exercise, the image of pressures of the feet, and a series of indicators with different angles of movement.

The computer software of the computer equipment processes the information provided by the video capture means and the baropodometric images provided by the pressure platform, provides the analytical data for the personalized biomechanical study of the individual, identifies biomechanical alterations that are the cause of a lesion or that can pose a risk of injury to the individual, and optionally designed a totally personalized treatment by means of templates for correcting the detected alterations.

Having sufficiently described the nature of the invention, as well as an example of a preferred embodiment, it is stated for the relevant purposes that the materials, shape, size and arrangement of the elements described may be modified, so long as this does not entail an alteration of the essential characteristics of the invention claimed below.

1. A system for a biomechanical study of a movement of an individual, the system comprising:
a treadmill including a pressure platform provided with a high-resolution pressure sensor, which obtains real-time high-resolution baropodometric images of an individual's tread while walking or running on a treadmill belt;
a video capturing device for capturing video in real time of a posture of the body of the individual during the practice of the exercise on the treadmill;
a computer device connected to: the video capture device and the pressure platform and a user interface for a specialist responsible for controlling the mode of execution of the software and the information of the software individual displayed on a screen; said computer equipment includes intelligent software that includes algorithms that:
process the information provided by the video capture device, obtains data from positions, angles, speeds and accelerations of the movements of the individual in the three planes of the space and compares them to ranges of values pre-determined, considered normal, deficient, optimal and pathological, determining those movements of the individual that come out of normal;
process the baropodometric images provided by the pressure platform, dynamically analyzes the pressure distribution over time and with the entire extracted tread sequence, discards the rams that do not follow the normal pattern of the sequence, and obtains a medium pressure distribution models; and
provides the analytical data for the personalized biomechanical study of the individual, identifying biomechanical alterations that are the cause of a lesion, or which can pose a risk of injury to the individual, and optionally designed a totally personalized treatment by templates suitable for correcting the detected alterations.

2. The system according to claim 1, wherein the real-time video capture cameras comprise one or more RGB-D vision sensors capable of measuring the depth to which the image objects are.

3. The system according to claim 1, wherein the real-time video capture cameras comprise one or more fixed and calibrated sensors.

4. The system according to claim 1, wherein the video capture cameras are suitable for delivering from 60 to 90 fps.

5. The system according to claim 1, further comprising a user interface for the individual, showing the video and the posture, the image of pressures of the feet, and a series of indicators with different angles of movement.

6. The system according to claim 1, wherein the image processing software provided by the vision sensors comprises an algorithm that registers 3D point clouds in a single point cloud and an optimization algorithm that extracts for each image the skeleton, and eventually applies a smoothing technique, post-processes the sequence of skeletons and obtains the data concerning the movements: positions, angles, linear or angular velocities and linear or angular accelerations in the three planes of space.

7. The system according to claim 1, wherein the baropodometric image processing software comprises a local optimization algorithm at subpixel level that temporarily aligns the sequence of such baropodometric images, determines the displacement between consecutive frames; and extracts the historical image of the footfalls, displaying the entire temporal sequence in space.
